(19) United States
(12) Patent Application Publication
(10) Pub. No.: US 2021/0042695 A1
(43) Pub. Date: Feb. 11, 2021

(54) Characterizing Medication Container Preparation, Use, and Disposal Within a Clinical Workflow

(71) Applicant: CRISI Medical Systems, Inc., Franklin Lakes, NJ (US)

(72) Inventors: Stephen M. Prince, La Jolla, CA (US); Walter J. Bochenko, Encinitas, CA (US); Christopher Biagioli, La Jolla, CA (US)

(21) Appl. No.: 17/081,767

(22) Filed: Oct. 27, 2020

Related U.S. Application Data
(63) Continuation of application No. 13/549,278, filed on Jul. 13, 2012.
(60) Provisional application No. 61/507,540, filed on Jul. 13, 2011.

(51) Int. Cl. G06Q 10/08; G06Q 10/10; G06Q 10/06; G16H 20/13; G16H 40/20

(52) U.S. Cl. CPC G06Q 10/0833; G06Q 10/103; G06Q 10/087; A61M 2205/60; G16H 20/13; G16H 40/20; G06Q 10/0633

(57) ABSTRACT

Data characterizing a medication container (e.g., an identifier, etc.) is received from a manually administrable medication device within a clinical workflow. Thereafter, one or more data records are generated, modified, or appended to include a portion of the received data. Subsequent requests that include the medication source from remote sources are answered by transmitting data stored in the one or more data records. Related apparatus, systems, methods, and articles are also described.

### CROSS-REFERENCE TO RELATED APPLICATIONS

This application is a continuation of U.S. patent application Ser. No. 13/549,278 filed on Jul. 13, 2012, which claims priority to U.S. Pat. App. Ser. No. 61/507,540 filed on Jul. 13, 2011, the contents of which are hereby fully incorporated by reference. In addition, this application relates to each of the following applications, which are all entitled “Medication Injection Site and Data Collection System”: U.S. patent application Ser. No. 12/938,300 filed on Nov. 2, 2010; U.S. patent application Ser. No. 12/765,707 filed on Apr. 22, 2010; U.S. patent application Ser. No. 12/614,276 filed on Nov. 6, 2009, and “Medication Waste and Data Collection System”: U.S. patent application Ser. No. 13/170,073 filed on Jun. 27, 2011, “Medication and Identification Information Transfer Apparatus”: U.S. patent application Ser. No. 12/768,509 filed on Apr. 27, 2010, “Medication Dose Preparation and Transfer System”: U.S. patent application Ser. No. 13/524,736 filed on Jun. 15, 2012, and “Selectively Controlling Fluid Flow Through a Fluid Pathway”: U.S. patent application Ser. No. 13,529,876 filed on Jun. 21, 2012. The contents of all of these applications are hereby fully incorporated by reference.

### FIELD

The subject matter described herein relates to characterization, preparation, use, and/or disposal of IV (intravenous) medication fluid containers with identifiers used with at least one manually administrable medication device within a clinical workflow.

### BACKGROUND

Injectable medications and fluids are frequently utilized by healthcare providers (caregivers) in the care of patients in the hospital, in pre-hospital emergency medical services (EMS) and at alternate care sites (including skilled nursing facilities, home health and hospice settings). Caregivers can include medical doctors, registered nurses, EMS paramedics, dentists and other licensed healthcare practitioners. Accurate documentation of what, when and how much medication and/or IV fluid is given to a patient is required by healthcare institutions, governmental agencies and regulatory oversight agencies. This is especially true when the IV fluid being administered to the patient is a medication or blood product.

Many health care procedures involve medication and other IV fluid administrations. The type of IV fluid and timing of administration are important to record in order to provide healthcare providers real-time information on the conduct of the procedure and the completion of a medical record. Some protocols require quick IV fluid administrations with limited time for documentation and record keeping. Others require completion and verification of IV fluid administration manually to ensure proper patient care and accounting for use of IV fluids.

Many IV fluid medications are controlled substances which require additional levels of controlled access, accurate administration documentation and the secure disposal of unused medication and medication containers. Tracking of medication amounts and containers can become difficult and time consuming.

Most IV fluids are packaged for adults and frequently patients are administered less than the full amount of fluid in an IV fluid container, resulting in unusable fluid that becomes waste. For example, there is almost always waste in pediatric drug dispensing because most drugs are packaged for adult doses. The process of disposing and documenting controlled substances as waste consumes time and resources. Most practitioners would agree that verifying the dose of a controlled substance prior to administration is a practice standard that should be upheld. But the second part of the verification, verifying remaining IV fluid in a vial, ampoule, bag, syringe or other IV fluid container with residual unused IV fluid to be disposed of as waste is a step identified as burdensome and is sometimes neglected by busy practitioners who seek ways to circumvent the process. In addition, the controlled substance disposal process may also be deliberately violated in a effort to divert (i.e. steal) controlled drugs for personal consumption or resale. Disposing of all controlled substances should follow rigorous procedures, but without requiring unnecessary steps for a caregiver and witness to verify actual wastage. What is needed is a simple and easy process to follow that still includes rigorous tracking and reporting capabilities.

Additionally, there are a number of patient clinical settings that require transfer of IV fluids from original manufacturer's primary containers to secondary containers to facilitate caregiver preparation and administration to patients. When IV fluids are transferred from primary containers to secondary containers it is standard clinical best practice to label them to reduce the potential for fluid administration errors. However, the incorrect transfer of labeling information and other factors may cause errors to continue to occur when caregivers transfer IV fluids from primary containers to secondary containers. For example, medications provided in vials are transferred to empty syringes; medications provided in prefilled syringes are transferred to empty syringes; clinicians prepare partial doses where vials or prefilled syringes are partially transferred to empty syringes or medications are diluted by transferring diluent fluids into syringes (where a partial amount of medication is withdrawn from a primary container or is further diluted into a secondary container). Tracking of IV fluid amounts and containers can often become difficult and time consuming.

For the purposes of safety, providing quality care delivery, compliance, and documentation, tracking the IV fluid delivery process is important to ensure the right clinicians are delivering the right IV fluid, to the right patient, in the right dose, at the right time; and, in the case of controlled substances, that residual IV fluid medications are completely and properly disposed of. This involves manual and/or automated tracking systems that link various sources of information including but not limited to clinician ID's, patient ID's, equipment ID's, IV fluid types, times, and doses. The data set characterizing a given fluid administration grows through the process from start to finish, with the data at one stage of the fluid delivery process often informing further stages of the process. For example, if Clinician A dispenses Narcotic B at the start of the process and administers half the dispensed quantity to a patient, then it is known that at the end of the process a step must occur where Clinician A properly disposes of one-half dose of Narcotic B (the remaining residual in the dispensed container) in the presence of a second clinician.

### SUMMARY

In one, aspect an IV fluid container identification system is provided that can include an IV fluid container with at least one unique identification element. A unique identification element can be located proximate to or on the fluid outlet of the IV fluid container or in other positions on the IV fluid container. The identification element can be attached to the IV fluid container at any step in the IV fluid delivery process including, but not limited to: during original manufacturing; during an external re-packaging operation (e.g. external pharmacy compounding); within a hospital pharmacy; at a medication or supply dispensing station; or, by a caregiver at the point of care during or just prior to an IV fluid preparation, IV fluid delivery, or as part of IV fluid waste disposal.

The identification element can be a label (printed on or applied to) on the medication container or a disk or ring attached to or surrounding the fluid outlet of the IV fluid container. The identification element can have an opening larger than a diameter of the fluid outlet tip of an IV fluid container (syringe or other containers) and smaller than or equal to the diameter of the barrel portion. In other variations the identification element can be slightly larger in diameter than the barrel portion, if the fluid container is a syringe. The identification element can contain optical, magnetic, RFID (radio frequency identification device), electronic and/or mechanically encoded information. Information on the identification element can include a unique identifier (such as a sequential serial number, a random or semi-random ID identifier (alpha-numeric sequence, hexadecimal code with-or-without a prefix, suffix, code base subscript number} or other unique information data, prefix, suffix, symbol or color, etc.). This information can be used to identify the IV fluid container and provide for tracking of it throughout the IV fluid delivery process. The information can be patient-specific or patient-neutral.

The identification element can be identified (read) by a sensor located in a fluid delivery system or any other device and/or system associated with the IV fluid delivery process which can include hand-held barcode readers, automated medication dispensing cabinets, label printing devices and infusion pumps. The sensor can be proximate to or on a fluid delivery inlet of the fluid delivery system. The information within the identification element can be readable by an identification sensor when the identification element is located around the fluid outlet tip of the medication container and the IV fluid container is coupled to or adjacent to a fluid delivery system to deliver contents of the IV fluid container. The information element can be located elsewhere on the body of the IV fluid container.

The identified IV fluid container can be provided empty or pre-filled with an IV fluid such as medication. An IV fluid container can be a syringe, a vial (single dose or multi-dose), an ampoule, a bag, a pouch, a bottle, a disposable cartridge, or a rigid or semi-rigid container.

It should be appreciated that use of the term “fluids” herein is not limited to a specific fluid type, therapy or medication and can include a variety of appropriate fluids. The use of the word “fluids” includes medications and other fluids for parenteral administration to a patient. The use of the word “medication” is intended to include any and all IV fluids. Fluids as used herein can include, but are not limited to medications, blood-based products, nutritional solutions, electrolytes, buffer solutions, lactated Ringer's solutions, sodium bicarbonate, crystalloids, colloids, saline solutions. Blood-based products can include, but are not limited to, any component of the blood for use in blood transfusions, whole blood, fresh frozen plasma, cryoprecipitate, blood substitutes, artificial blood, oxygen-carrying substitutes. Medications can include any therapeutic fluid that can be administered intravenously or another appropriate parenteral route of administration such as intra-arterial, intraosseous, intracerebral, intracardiac, subcutaneous, or intraperitoneal. Similarly, the systems described herein are not limited to a specific IV fluid source container type and can include syringes, IV bags, disposable medication cartridges or pouches, infusion pumps, and IV tubing, to name a few examples.

It should further be appreciated that use of the term “characterizing the medication container” herein is not limited to data associated with the physical container itself (unless otherwise specified), but also includes data relating to its contents and any activities, processes and/or workflows involving the medication container's manufacturing and/or use.

The identified container can be used for tracking activities during its manufacturing, shipping, repackaging, and/or external compounding phases of container distribution; and/or throughout the preparation, administration, and documentation phases of its clinical use within a healthcare delivery environment. The identified IV fluid container can be identified (tracked) at any one or more of: a container production facility, an external pharmacy compounding facility, a hospital pharmacy, a blood bank, a medication or supply dispensing station, during IV fluid preparation and/or transfer to a secondary container, during IV fluid administration to a patient, or during IV fluid waste disposal done before or after IV fluid administration to a patient. The identified container tracking can be for the purpose of patient specific information management, patient billing, IV fluid administration records, IV fluid waste disposal, process/workflow improvement, facilitation of logistics, ensuring patient safety measures are followed, compliance with healthcare institution procedures, compliance with healthcare agency rules and regulations, and/or compliance with governmental rules and regulations.

The information within the identification element can be transmitted to a data collection system for recordkeeping. The data collection system can be any one or more of: an electronic medical records system, a medication administration records system, a pharmacy system, a blood bank information system, an IV fluid storage and dispensing system, an IV fluid waste collection and disposal system, or a logistics tracking system.

Devices or systems that read the identification element on the IV fluid container can access, act upon and/or modify the data set pointed to by the information element. The data set on the data collection system can be dynamic with new data elements added by devices and systems that interact with the IV fluid container as the fluid delivery process progresses from start to finish. For example, when a medication is initially dispensed, the automated dispensing system it is removed from can add data elements such as medication type, concentration, container type, initial container volume, dispensing clinician ID, and/or dispensing time to the data set pointed to by a unique identifier within the information element on the dispensed IV medication container.

The data set on the data collection system produced as the IV fluid container information element interacts with devices and systems during the fluid delivery process can be used for medical record documentation, process tracking, inventory control, drug diversion prevention, quality control and statistical analysis, billing, or any other clinical or operation application of the data elements collected.

In a further aspect, data is received from a manually administrable medication device that characterizes a medication container. The manually administrable medication device is one of a plurality of medical devices used within a clinical workflow and the medication container comprises an identifier. In response to receiving the data, at least one data record is generated, modified and/or appended with at least a portion of the received data using the identifier (e.g., the identifier may be used to generate new records and/or the identifier may be used to identify pre-existing records, etc.). A request that includes the medication identifier is later received from a remote source. In response to the request, data stored within the at least one data record associated with the medication container is transmitted to the remote source.

The manually administrable medication device can be a device to characterize and may involve the manual actions of one or more individuals relating to filling or preparing the filling of medication into the medication container, dispensing or preparing to dispense medication from the medication container (including administration to the patient), and wasting or preparing to waste medication from the medication container.

The remote source that generated the request can be the manually administrable medication device that generated the received data. In addition, the remote source that generated the request can be one or more of the medical devices in the workflow other than the manually administrable medication device that generated the received data.

The remote source when receiving the transmitted data can display at least a portion of the transmitted data, provide audio and/or visual feedback relating to at least a portion of the transmitted data, and/or apply at least one decision rule using at least a portion of the transmitted data as input. With the latter, the decision rule can be used to determine how to provide care for a patient, characterize care given to the patient, and/or how to operate one or more medical devices within the clinical workflow.

The remote source can be any device or system that consumes data generated by the manually administrable medication device and/or data stored in the at least one data record. Sample remote sources include, but are not limited to: pharmacy information systems, medication administration record systems, blood bank information systems, patient admissions record systems, electronic medical record systems, medical record documentation systems, anesthesia information management systems, operating room information systems, patient scheduling systems, barcode medication administration systems, barcode verification systems, clinical information systems, infusion pumps, patient-controlled analgesia systems, patient monitoring devices, automated medication dispensing systems, medication dispensing carts, automated supply cabinets, medication container filling units, medication compounding units, fluid composition sensors, medication preparation and transfer units, medication injection sites, intelligent fluid flow stops, medication waste and data collection systems, clinical procedure process tracking systems, inventory control systems, logistical tracking systems, drug diversion prevention systems, quality control measurement systems, statistical analysis systems, billing systems, and compliance verification systems.

The plurality of medical devices can include a wide variety of devices, including, but not limited to: a medication container filling unit, a medication compounding unit, a fluid composition sensor, an automated medication dispensing system, a medication dispensing cart, an infusion pump, a patient-controlled analgesia system, a medication preparation and transfer unit, a barcode medication administration system, a medication injection site, an intelligent fluid flow stop, and a medication waste collection system.

The received data can be any type of data generated or otherwise obtained/stored by the manually administrable medication device including, without limitation, a type of medication contained within the medication container, the concentration of medication contained within the medication container, a type of medication container, a maximum volume capacity of the medication container, a volume of medication contained within the medication container, a volume of medication extracted from the medication container, a volume of medication and/or diluents added to the medication container, a volume of medication manually administered from the medication container, a patient identifier, a caregiver identifier, a pharmacist identifier, a care area identifier, a pharmacy identifier, a device and/or system identifier, a medical order identifier, a primary container identifier, a secondary container identifier, a controlled substance identifier, at least one time stamp identifying the timing of an event within the clinical workflow, at least one medical procedure associated with the workflow, a medication expiration date, a dosage form of the medication, dose instructions for the medication, specific-patient administration instructions for a medication, a medication formulation, medication manufacturer information, a re-packager of the medication, a distributor of the medication, a medication package form, a medication package size, a medication container serial number, a medication lot number, a blood type of a patient, an NDC code (National Drug Code), an RxNorm code, a segment of an NDC code identifying a corresponding medication product, a segment of an NDC code identifying a corresponding medication package, a unique identifier code, a serialized NDC (sNDC) code, a drug classification, a human readable alphanumeric string, and a machine readable code.

The manually administrable medication device can read the medication container identifier. The manually administrable medication device can automatically read the medication container identifier when the medication container is coupled or in the process of being coupled thereto. The received data can be transmitted by the manually administrable medication device automatically upon reading of the medication container identifier (e.g., when the medication container is coupled or in the process of being coupled to the manually administrable medication device, etc.).

The remote source can be a manually administrable medication device (which may or may not be the manually administrable medication device that generated the received data), or an alternative type of remote source and the request can be transmitted by such remote source automatically upon reading of the medication container identifier (e.g. when the container identifier is read by a manually-operated barcode scanning device, etc.).

The identifier can be a unique number, a unique alphanumeric string, or a unique symbol. The medication container identifier can be linked to a secondary unique identifier, and such secondary unique identifier can be used to access or otherwise identify data stored in the at least one data record. The identifier can be a uniform resource locator (URL) (and such URL can be used by the remote source, etc.).

The medication containers can be, for example, syringes, intravenous (IV) bags, disposable medication cartridges, disposable medication pouches, single and multi-dose vials, ampoules, and IV tubing.

The various aspects can be implemented within a single computing system by one or more data processors or they can be implemented in a distributed environment utilizing two or more computing systems.

In some cases, complementary data associated with at least one of the medication container and medication contained within the medication container can be obtained by polling at least one data source. This complementary data can be used to generate, modify and/or append at least one data record. The complementary data can include data such as fluid information, patient-specific information, medical order information, clinical guideline information, environmental factors, and historical patient information, a type of medication contained within the medication container, the concentration of medication contained within the medication container, a type of medication container, a maximum volume capacity of the medication container, a volume of medication contained within the medication container, a volume of medication extracted from the medication container, a volume of medication and/or diluents added to the medication container, a volume of medication manually administered from the medication container, a patient identifier, a caregiver identifier, a pharmacist identifier, a care area identifier, a pharmacy identifier, a device and/or system identifier, a medical order identifier, a primary container identifier, a secondary container identifier, a controlled substance identifier, at least one time stamp identifying the timing of an event within the clinical workflow, at least one medical procedure associated with the workflow, a medication expiration date, a dosage form of the medication, dose instructions for the medication, specific-patient administration instructions for a medication, a medication formulation, medication manufacturer information, a re-packager of the medication, a distributor of the medication, a medication package form, a medication package size, a medication container serial number, a medication lot number, a blood type of a patient, an NDC code (National Drug Code), an RxNorm code, a segment of an NDC code identifying a corresponding medication product, a segment of an NDC code identifying a corresponding medication package, a unique identifier code, a serialized NDC (sNDC) code, a drug classification, a human readable alphanumeric string, and a machine readable code.

The manually administrable medication device can include a housing; a medication port extending from an outer surface of the housing to couple to a fluid outlet of the medication container, the medication port being fluidically coupled to a patient such that medication manually extracted from the medication container is immediately administered to the patient; an identification sensor disposed within the housing to generate information indicative of contents of the medication container when the fluid outlet of the medication container is fluidically coupled to, or in the process of being fluidically coupled to, the medication port; and a transmitter disposed within the housing and in communication with the identification sensor to wirelessly transmit the information generated by the identification sensor to a remote data collection system. In some implementations, a flow sensor can also be included to characterize a volume of fluid dispensed and/or administered to the patient (and the transmitter also transmits data characterizing same). The housing can have a shape and size enabling it to be held by a first hand of a user while the user administers medication from the medication container via the medication port using his or her second hand.

The manually administrable medication device can be or be part of a waste collection system to receive unused medication within the medication container for disposal. The waste collection system can include at least one sensor to generate data to identify and quantify an amount of medication received by the at least one waste collection system and to identify the medication container housing the medication. The waste collection system can include a transmitter for transmitting the received data.

The manually administrable medication device can be an apparatus for transferring medication from a primary medication container to a manually administrable secondary medication container. The secondary medication container can correspond to the medication container identified by the identifier in the received data. Such apparatus can include a fluid channel terminating at a primary medication container port on a first end and a secondary medication container port on a second end; a primary medication container coupling configured to fluidically couple the primary medication container to the primary medication container port; a secondary medication container coupling configured to fluidically couple the secondary medication container to the secondary medication container port; at least one identification sensor to sense (i) an information transfer element on the primary medication container and (ii) an information transfer element on the secondary medication container, the information transfer element on the primary container being used to characterize the medication; and a communications module to transmit data obtained by and/or derived from the at least one identification sensor to a remote computing system to enable the remote computing system to associate data characterizing the medication with the secondary medication container.

In some variations, the manually administrable medication device can be a medication container preparation device that is configured to receive data characterizing medication within a medication container, generate an identifier encapsulating data characterizing the medication, and apply the identifier to the medication container, the identifier being positioned such that it is automatically readable by a medication administration device when at least a portion of the medication is administered to a patient and/or a medication wasting device when at least a portion of the medication is wasted.

The manually administrable medication device can include: a fluid inlet configured to couple to an outlet of the medication container, the medication container having fluid source information encoded thereon; a fluid outlet configured to deliver fluid from the medication container to a fluid line leading to a patient; a fluid flow stop disposed between the fluid inlet and the fluid outlet that prevents fluid flow in a first state and permits fluid flow in a second state; an identification sensor to detect the fluid source information when the medication container is being coupled or is coupled to the fluid inlet; and a flow state controller to selectively cause the fluid flow stop to transition between the first state and the second state based on the fluid source information detected by the identification sensor.

In an interrelated aspect, first data is received from a first manually administrable medication device that includes an identifier identifying a medication container, an amount of medication administered from the medication container to a patient, and a timestamp corresponding to a time when the medication was administered to the patient. The first manually administrable medication device is one of a plurality of medical devices used within a clinical workflow and the medication container comprises an identifier. Thereafter, at least one data record is generated, modified, or appended with at least a portion of the received first data using the identifier. Second data is later received from a second manually administrable medication device. The second data includes the identifier, an amount of remaining medication within the medication container dispensed from the medication container into a wasting station subsequent to the administration of the medication to the patient, and a timestamp corresponding to a time when the medication was dispensed into the wasting station. In response, the at least one data record is generated, modified, or appended with at least a portion of the received second data. Subsequent requests from remote sources that include the medication identifier can be responded to by transmitting data stored within the at least one data record. In some related variations, third data can be received from a third manually administrable medication device prior to receiving the first data that includes the identifier and an amount of medication placed within the medication container. Such third data can also be used to generate new data records or modify or append existing data records.

In an interrelated aspect, data characterizing a medication container is received from a manually administrable medication device that is one of a plurality of medical devices used within a clinical workflow. Thereafter, at least one data record is generated, modified, and/or appended with at least a portion of the received data. Subsequently, a request comprising data associated with the medication container is received from a remote source. Data stored within the at least one data record associated with the medication container is then transmitted to the requesting remote source.

In a further interrelated aspect, data characterizing at least two medication containers is received from at least one manually administrable medication device. The at least one manually administrable medication device is one of a plurality of medical devices used within a clinical workflow that utilizes the at least two medication containers. Thereafter, at least one data record is generated, modified, and/or appended with at least a portion of the received data. One or more data record associates the two medication containers so that a request that includes data characterizing at least one of the medication containers from a remote source can be received. In response to such a request, data stored within the at least one data record associated with the at least one of the medication containers specified in the request is transmitted to the remote source.

Computer program products are also described that comprise non-transitory computer readable media storing instructions, which when executed by at least one data processor of one or more computing systems, causes the at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The details of one or more variations of the subject matter described herein are set forth in the accompanying drawings and the description below. Other features and advantages of the subject matter described herein will be apparent from the description and drawings, and from the claims.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed embodiments. In the drawings:

FIG. 1 is a diagram illustrating a medication container identification system;

FIG. 2 is a diagram describing a detailed view of a medication container identification system as in FIG. 1;

FIG. 3 is a diagram illustrating IV fluid delivery devices and systems that can interact with a container identification system;

FIG. 4 is a detailed diagram of a medication preparation and transfer system for use with a medication identification system as in FIG. 3;

FIG. 5 is a detailed diagram of a medication injection site for use with a medication identification system as in FIG. 3;

FIG. 6 is a detailed diagram of a second medication injection site for use with a medication identification system as in FIG. 3;

FIG. 7 is a detailed diagram of a waste collection system for use with a medication identification system as in FIG. 3;

FIG. 8 is a diagram illustrating a data collection and tracking system based on a medication identification system;

FIG. 9A depicts a process of preparing a primary IV container;

FIG. 9B depicts the removal of medication from an automated dispensing system (ADS);

FIG. 9C depicts the transfer of medication from the primary container to a secondary container;

FIG. 9D depicts administration of medication from the secondary container to a patient;

FIG. 9E depicts disposal or “wasting” of medication from an IV container (secondary or primary) to a waste collection system.

FIG. 10 is a diagram further illustrating a data collection and record keeping system as in FIG. 8.

FIG. 11 is a first process flow diagram illustrating storage of data received from a manually administrable medication device and transmission of data to a requesting remote source.

FIG. 12 is a second process flow diagram illustrating storage of data received from a manually administrable medication device and transmission of data to a requesting remote source.

Like reference symbols in the various drawings indicate like or similar elements. As indicated previously, the use of the words “medication” and “IV fluid” are used interchangeably in the following description.

### DETAILED DESCRIPTION

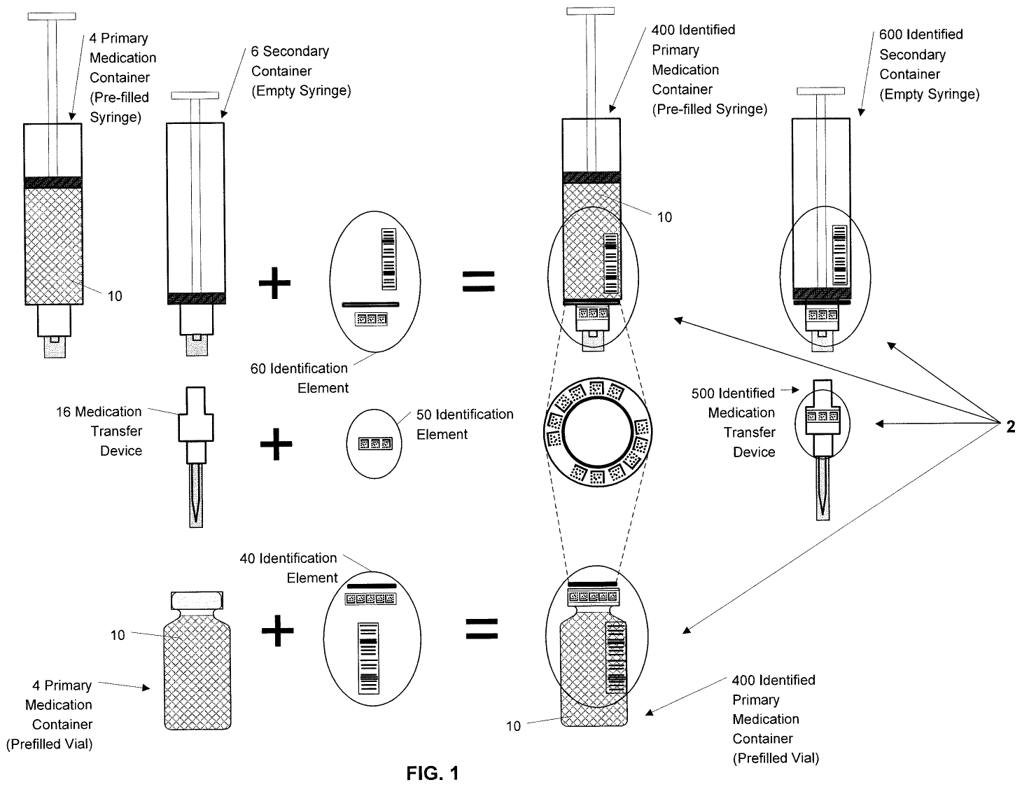

FIG. 1 is a diagram illustrating a medication container identification system 2. Primary medication containers 4 (pre-filled syringes, pre-filled vials, pre-filled rigid/semi-rigid/flexible containers with or without fluid delivery tubing) can contain medication 10 as provided by pharmaceutical companies. Conventional labeling on medication containers 4 can include the manufacturer's pharmaceutical information including medication name, concentration, NDC code, expiration date, volume enclosed, part number, precautions and other information. Secondary container 6 (empty syringe, empty or partially pre-filled fluid bags or semi-rigid/flexible containers) can be provided for fluid transfer prior to administration to a patient. Primary medication containers 4 can also include identification elements 40 (for vials) and 60 (for syringes) providing unique container information. Secondary containers can also include identification elements 40 (for vials) or 60 (for syringes) providing unique container information. Medication containers 4 with identification elements 40 and 60 can be uniquely identified as identified primary containers 400 (pre-filled syringes, pre-filled vials, pre-filled rigid/semi-rigid/flexible containers) and/or identified secondary containers 600 (empty syringes, vials, rigid/semi-rigid/flexible containers with or without fluid delivery tubing). These identified containers can be used for tracking the medication dispensing, preparation, administration and disposal of unused medication.

Medication transfer device 16 can be used to transfer medication from primary medication containers 4 to secondary medication containers 6. These transfer devices 16 can include needles as depicted and can access medications in pre-filled vials or syringes and facilitate manual fluid transfer from one container to another. Transfer device 16 can include an identification element 50 and form identified transfer device 500. In use, identified transfer device 500 can be separable allowing the needle access portion to remain with the primary container while the identifier element 50 remains attached to the secondary container identifying thus identifying the secondary container. Refer to “Medication and Identification Information Transfer Apparatus”: U.S. patent application Ser. No. 12/768,509 filed on Apr. 27, 2010.

The identification element 40, 50 or 60 (Identification code—ID Code) can be a unique number, code, symbol, serial number, random number, or other information describing a specific unique medication container or transfer device 400, 500 or 600 respectively. A medication dose can be manually transferred from primary container 4 through medication transfer device 16 into secondary container 6 by pulling on the plunger rod of syringe 6 and drawing medication dose 10 into it.

The ID Codes 40, 50 or 60 can be applied by any number of operations or steps in the supply chain of medication containers 4 or 6 prior to medication transfer including, but not limited to: the original primary or secondary container manufacturer, a pharmaceutical re-packager, a hospital pharmacy, a healthcare professional or caregiver, a patient. ID Codes 40, 50 or 60 can be one or more of an optical source, a magnetic source, a mechanical source, a switchable RFID source, a conductive source, and/or a proximity source. One implementation can provide information encoded within the identification element in the form of an optically detectable surface, reflective or absorbing light surface, and can be embedded into or on top of the element body.

Alternatively, information provided by ID Codes 40, 50 or 60 can be a magnetically detectable strip similar to a credit card magnetic strip, facilitating a magnetic scan similar to credit card swiping, that is embedded into or on top of the identification element body.

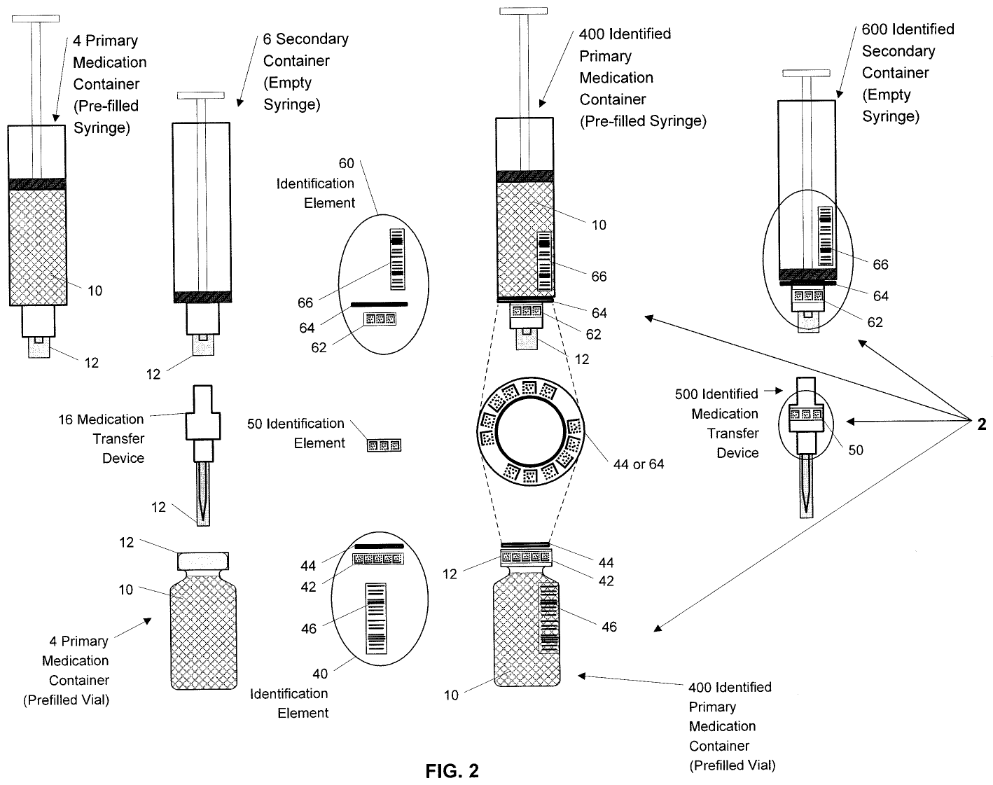

FIG. 2 is a diagram describing a detailed view of a medication container identification system 2 as in FIG. 1. Primary medication container 4 (vial) can have an identification element 40 in any of a number of locations upon container 4 to form identified primary container 400. Identification element 42 can be affixed to the side portion of the vial closure, identification element 44 can be affixed to the top of the vial closure or Identification element 46 can be affixed to the side of the vial body. Other positions can be envisioned on other surfaces of the vial.

Primary medication container 4 (syringe) can have an identification element 60 in any of a number of locations upon container 4 to form identified primary container 400. Identification element 62 can be affixed proximate to or on the fluid outlet, identification element 64 can be a disk or hub located concentric to the fluid outlet, or Identification element 66 can be affixed to the side of the syringe body. Other positions can be envisioned on other surfaces of the syringe.

Secondary medication container 6 (empty syringe) can have an identification element 60 in any of a number of locations upon container 6 to form identified secondary container 600. Identification element 62 can be configured proximate to or on the fluid outlet, identification element 64 can be a disk or hub located concentric to the fluid outlet, or Identification element 66 can be affixed to the side of the syringe body. Other positions can be envisioned on other surfaces on the syringe. Similarly, other secondary containers 6 (empty vials, rigid/semi-rigid/flexible containers with or without fluid delivery tubing) can have an identification element 60 in any of a number of locations upon the container to form identified secondary container 600. Identification element 62 can be affixed proximate to or on the fluid outlet, identification element 64 can be a disk or hub located concentric to the fluid outlet, or Identification element 66 can be affixed to the side of the container body. Other positions can be envisioned on other surfaces on the container.

Additionally, medication transfer device 16 can have an identification element 50 which can be affixed to one or more of several positions upon the medication transfer device 16 to form identified medication transfer device 500. Identification element 50 can be affixed proximate or upon the fluid outlet as a label, a disk or hub located concentric to the fluid outlet, or can be affixed on the side of the transfer device 16. Other positions for affixing element 50 can be envisioned on other surfaces of the medication transfer device 16.

Primary medication container (vial) 4 can be coupled to secondary container (syringe) 6 by medication transfer device 16 for manual fluid transfer. Vial 4 can be coupled (spiked) using medication container transfer device 16 attached to the fluid outlet of secondary container (syringe) 6. Transfer device 16 can contain a fluid transfer channel. Adapter 16 can be or include a vial adapter, a needle, a blunt tip cannula, a needle-less luer adapter with spike or any fluid transfer apparatus designed for the transfer of medication 10 from vial 4 to syringe 6.

Transfer device 16 can include an identification element 50 and form identified transfer device 500. In use, identified transfer device 500 can be separable allowing the needle access portion to remain with the primary container while the identifier element 50 remains attached to the secondary container identifying thus identifying the secondary container.

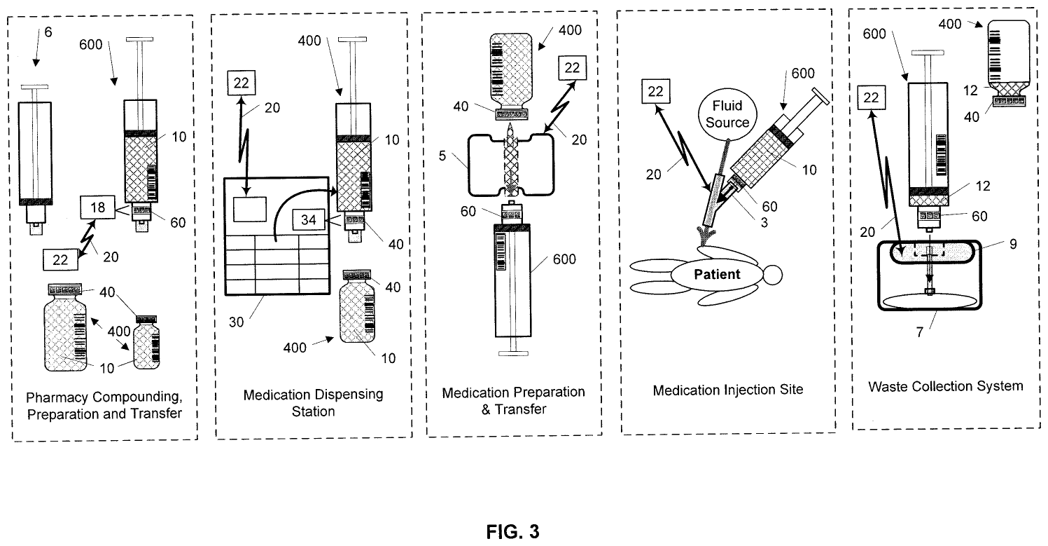

FIG. 3 is a diagram illustrating several uses of a medication container identification system 2 as in FIG. 2. System 2 can be used in any number of environments for the identification and tracking of medication containers. Starting from the left, a pharmacy or medication compounding service can use identification element 60 to indicate a diluted or compounded medication 10 and/or transfer of medication 10 to container 600. Transfer of medication can be manual or mechanized. Identification element 60 can be applied before or after dilution or compounding medication 10 and can be scanned or read by detector 18. Information can be transferred 20 to data collection system 22 recording the pharmacy activity on medication container 600. Pharmacy personnel performing the activity can be recorded and associated with ID Code 60 utilizing a database that is associated with or part of data collection system 22.

In the second from the left box, system 2 can be used at a medication dispensing station 30 (e.g. Pyxis® MedStation) or mobile medication cart (e.g. Pyxis® Anesthesia System) where medication container 400 can be dispensed from a drawer. A user can scan 34 identification element 40 to identify medication container 400. Information can be transferred 20 to data collection system 22 recording the manual medication dispense activity. Dispensing personnel performing the activity can be identified, recorded and associated with ID Code 40. A database that is associated with or part of data collection system 22 can provide this association.

In the center box, system 2 can be used at a medication preparation and transfer step where primary medication container 400 and secondary medication container 600 (empty syringe) can be used with a dose preparation device 5. Manually administrable medication device 5 can include an identification sensor that scans and can associate (or link) ID Code 40 with ID Code 60 and the associated (or linked) data that correlates ID code 40 to ID code 60 can be transmitted 20 to data collection system 22 for tracking purposes. Device 5 can have a housing with a shape and size enabling it to be held by a first hand of a user while a user manually transfers medication from the primary container to the secondary container with his or her second hand. Dose preparation personnel can be identified, recorded and associated with ID Code 40 and/or 60. Refer to “Medication Dose Preparation and Transfer System”: U.S. patent application Ser. No. 13/524,736 filed on Jun. 15, 2012. A database associated with or included in data collection system 22 can associate any combination or all of code 40, code 60, data preparation personnel identities, and other related information.

In the second from the right box, system 2 can be used at a patient's medication injection site step where medication container 600 (or 400 if a prefilled syringe) can be used with an intelligent injection site 3. Manually administrable medication device site 3 can scan ID Code 60 and data including data representing code 60 can be transferred 20 to data collection system 22 for tracking purposes. Injection site 3 can have a housing with a shape and size enabling it to be held by a first hand of a user while a user manually administers medication with his or her second hand. Dose administration personnel can be identified, recorded and associated with ID Code 60 utilizing a database associated with or included with system 22. Refer to “Medication Injection Site and Data Collection System”: U.S. patent application Ser. No. 12/938,300 filed on Nov. 2, 2010; U.S. patent application Ser. No. 12/765,707 filed on Apr. 22, 2010; and U.S. patent application Ser. No. 12/614,276 filed on Nov. 6, 2009.

In the right most box, system 2 can be used at a medication waste collection step where unused medication 12 can be disposed of from container 600 to a waste collection system 7. Waste collection system 7 can have a manually administrable medication device intelligent injection site 9. Site 9 can include a means of reading ID code 60 such as an optical or magnetic sensor. Site 9 can thereby read ID Code 60 and transfer (indicated by transmission element 20) data including ID code 60 to data collection system 22 for tracking purposes. Additionally or alternately, unused medication 12 can be manually disposed of from container 400 to intelligent injection site 9. Injection site 9 can read ID Code 40 and data including ID code 40 can be transferred 20 to data collection system 22 for tracking purposes. Waste disposal personnel can be identified, recorded and associated with ID Code 40 or 60. A database associated with or part of system 22 may store and correlate information representing ID codes 40 and 60 as well as the identities of the personnel. Refer to “Medication Waste and Data Collection System”: U.S. patent application Ser. No. 13/170,073 filed on Jun. 27, 2011.

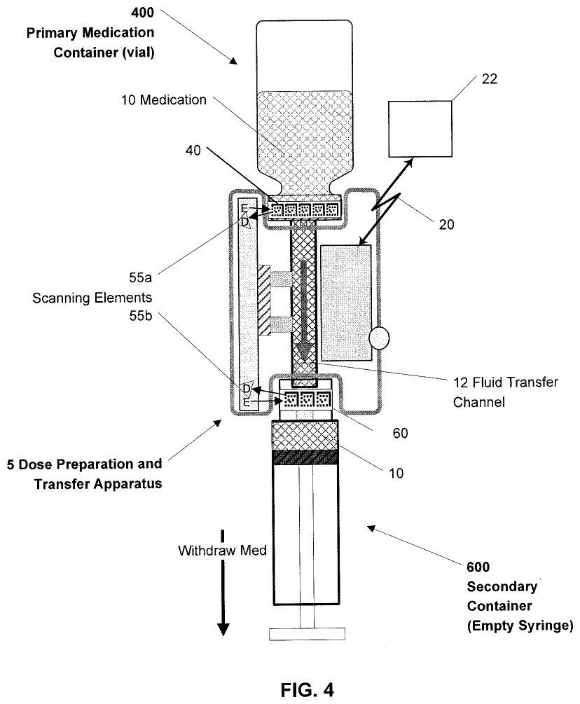

FIG. 4 is a detailed diagram of a medication preparation and transfer system 5 for use with medication identification system 2 as in FIG. 3. Primary medication container 400) can be provided with identification element 40 on prefilled vial 4 or identification element 60 on prefilled syringe 4 (not shown) to uniquely identify the primary container 400. Additionally, secondary medication container 6 (an empty syringe) can be provided with an identification element 60 to uniquely identify the secondary container 600. Identification element 40 can be configured as an identifying label 42 located proximate to or on the fluid outlet of the primary container, an identifying disk 44 proximate to the primary container closure or an identifying label 46 on the body of the primary container as shown in FIG. 2. Identification element 60 can be configured as an identifying label 62 located proximate the fluid outlet of the primary container, an identifying disk 64 proximate the primary container cap or an identifying label 66 on the body of the primary container as shown in FIG. 2.

The fluid outlet of medication container 400 can be attached to the fluid inlet end of fluid transfer channel 12. When attached, scanning or sensor element 55a can read or identify identification element 40. Similarly, the fluid outlet end of fluid transfer channel 12 can be attached to secondary container 600. When attached, scanning or sensor element 55b can read or identify identification element 60. Once read, identification elements 40 and 60 can be associated (or linked) with each other. Medication 10 in container 400 can be transferred (withdraw med) to container 600 by pulling on the plunger rod of container 600. A fluid transfer measurement can be made to record the amount of medication transferred to container 600. The identified information 40 and 60 and the amount of medication transferred can be transmitted 20 to data collection system 22 for tracking purposes. Dose preparation personnel can be identified, recorded and associated with ID Code 40 or 60 within a database that is associated with or forms part of data collection system 22.

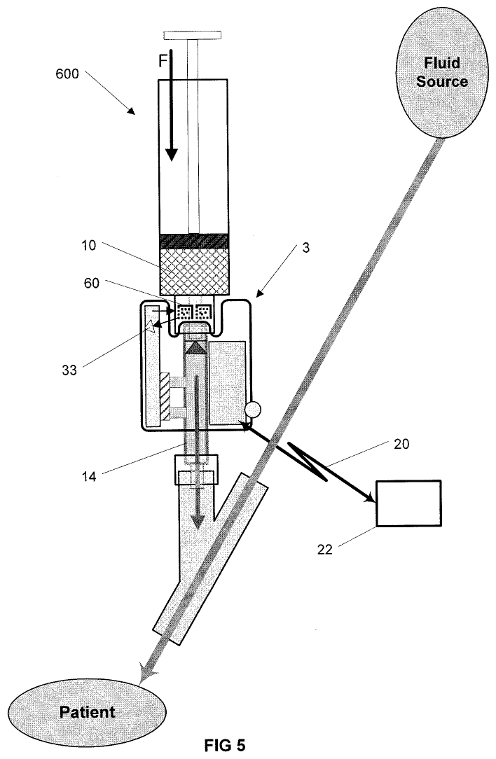

FIG. 5 is a diagram describing a detailed view of a medication injection site 3 using a medication container identification system 2 as in FIG. 3. Medication injection site 3 can be used for the administration of medications to a patient. A fluid source can be attached to fluid delivery tubing with a “Y” site proximal the patient. Medication container 600 can have identification element 60 to be identified by injection site 3. Medication container's 600 fluid outlet can be configured to connect to injection site's 3 fluid inlet for fluid injection (administration to the patient). Injection site 3 can have detection and/or scanning sensor element 33 to identify identification element 60. When the fluid junction is made, scanning element 33 can identify identification element 60.

When medication is manually administered (indicated by force F applied to syringe plunger rod in FIG. 5) a sensor within injection site 3 can sense an amount of fluid transferred from container 600 through the site 3. Site 3 can then transmit information (indicated by element 20 in FIG. 5) to data collection system 22 that represents identification 60 and the amount of medication administered. A database associated with or part of system 22 can associate identification 60, the amount of medication administered, and an identity of administration personnel.

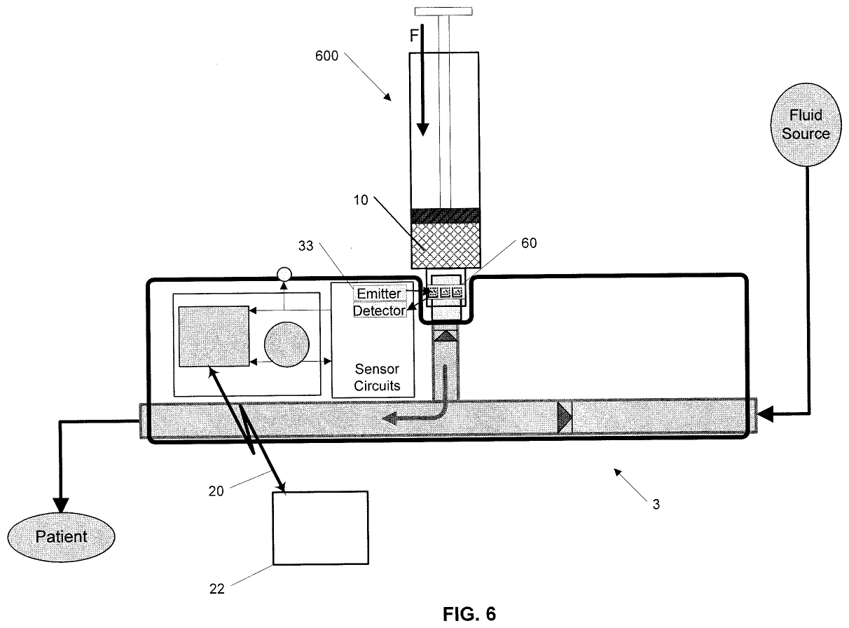

FIG. 6 is a detailed diagram of a second medication injection site 3 for use with a medication identification system 2 as in FIG. 3. Medication injection site 3 can be used for the manual administration of medications to a patient. A fluid source can be attached to fluid delivery tubing attached to the patient. Injection site 3 can be joined to the fluid pathway tubing by a secondary fluid channel for the administration of injections. Medication container 600 can have identification element 60 to be identified by injection site 3. Medication container's 600 fluid outlet can be configured to connect to injection site's 3 fluid inlet for fluid injection (administration to the patient). Injection site 3 can have detection and scanning sensor element 33 (emitter/detector) to identify identification element 60. When the fluid junction is made, scanning element 33 can identify identification element 60. Once scanned, identification element 60 can be associated with manual medication administration (push on the syringe plunger rod) to the patient and a fluid transfer measurement can be made to record the amount of medication administered to the patient. The identification information 60 and the amount of medication administered can be transmitted 20 to data collection system 22 for tracking purposes. Medication administration personnel can be identified, recorded and associated with ID Code 60.

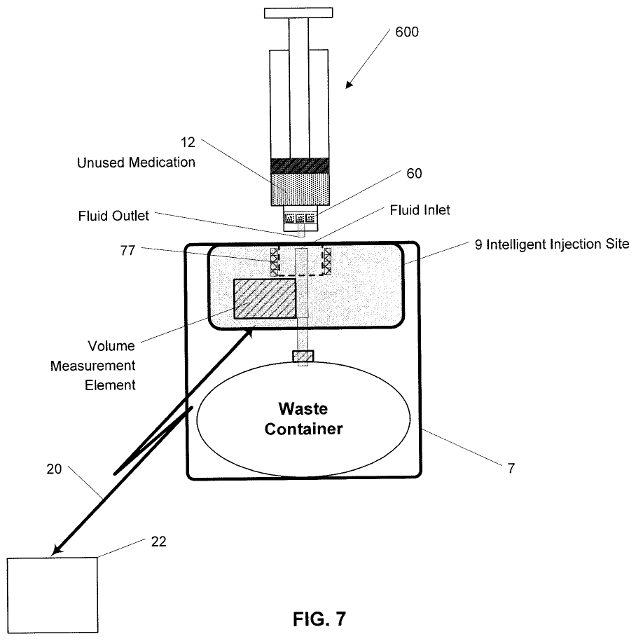

FIG. 7 is a diagram describing a detailed view of a medication waste collection system 7 site using a medication container identification system 2 as in FIG. 3. Waste collection system 7 can have an intelligent injection site 9 used for the disposal of unused medication 12. Injection site 9 can be joined to a waste container by tubing. Medication container 600 can have identification element 60 to be identified by injection site 9. Medication container's 600 fluid outlet can be configured to connect to injection site's 9 fluid inlet for manual fluid disposal (medication waste). Injection site 9 can have detection and scanning sensor element 77 (emitter/detector) to identify identification element 60. When the fluid outlet of medication container 600 is connected to the fluid inlet of injection site 9, scanning element 77 can identify identification element 60. Once scanned, identification element 60 can be associated with the medication disposal. A fluid disposal measurement can be made to record the amount of disposed or discarded medication. The identification information 60 and the amount of medication disposed can be transmitted 20 to data collection system 22 for tracking purposes. Medication disposal personnel can be identified, recorded and associated with ID Code 60. A database associated with or part of data collection system 22 can correlate the identification information, the amount of medication discarded, and personnel performing or associated with the medication disposal.

Other steps can be envisioned and can be included at any point in the medication delivery cycle and by any healthcare professional. These steps can be any one or more of the following, but are not limited to: filling primary medication containers with medication at the drug manufacturer, mixing of medications, compounding of medications, dilution of medications, transfer of medications from single dose primary containers to secondary containers, transfer of medications from multi-dose primary containers to multiple secondary containers, partial administration of medications to patients, administration of solutions from bags or bottles, admixture transfer of medications to fluid source bags or bottles, connection of fluid source bags/bottles to fluid delivery tubing, and connection of fluid delivery tubing to injection sites to name a few examples.

Further and alternatively, information provided by the information element 40, 50 or 60 can be a mechanically detectable feature consisting of Braille like features of bumps or ridges or valleys or peaks on the surface of or at the end of element body, facilitating mechanical detection by a micro switch or similar physical detection method. Further and alternatively, information provided by ID Code 40, 50 or 60's information element can be an RFID (radio frequency identification device) tag located on the surface of element body, facilitating detection by an RFID reader. The antenna of the RFID tag can be switchable and would be OPEN prior to connection to the medication dose preparation and transfer system 5, injection site 3 or waste collection injection site 9. Upon connection of the medication container fluid outlet to the inlet of the transfer system 5 or injection site 3 or 9 the antenna can become CLOSED (or connected) facilitating RFID reader detection. When the medication container is disconnected the RFID tag antenna can again become OPEN.

Further and alternatively, information provided by ID Code information element 40, 50 or 60 can be in the form of a capacitive or inductive proximity feature on the surface of or embedded into element body, facilitating capacitive or inductive proximity detection.

ID Code information element 40, 50 or 60 can be an integrated feature of the information transfer element such as etched or molded features. The information element can alternatively be adhered or deposited to the element body (i.e., information element can be a label, etc.) or embedded therein. In addition, the information element 40, 50 or 60 can be a separate element that extends around fluid outlet.

In some implementations, an intelligent fluid flow stop can be utilized such as that described in co-pending application U.S. patent application Ser. No. 13,529,876 entitled “Selectively Controlling Fluid Flow Through a Fluid Pathway”. Such an intelligent fluid flow stop can include, for example: a fluid inlet configured to couple to an outlet of a medication container having fluid source information encoded thereon; a fluid outlet configured to deliver fluid from the medication container to a fluid line leading to a patient; a fluid flow stop disposed between the fluid inlet and the fluid outlet that prevents fluid flow in a first state and permits fluid flow in a second state; an identification sensor to detect the fluid source information when the medication container is being coupled or is coupled to the fluid inlet; and a flow state controller to selectively cause the fluid flow stop to transition between the first state and the second state based on the fluid source information detected by the identification sensor.

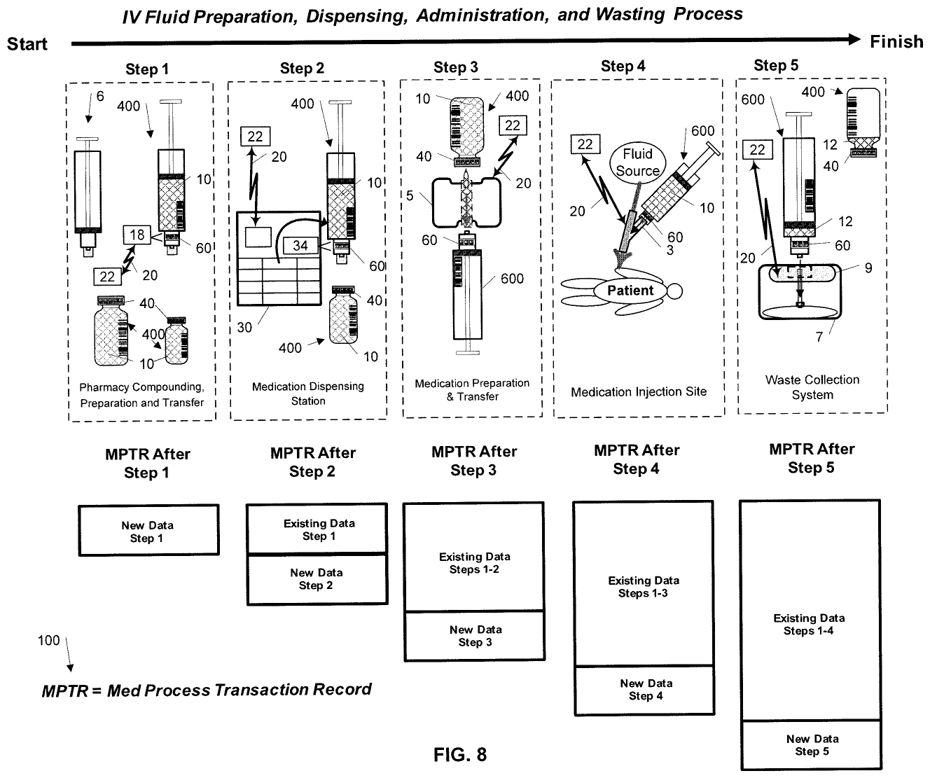

FIG. 8 is a diagram illustrating a data collection and tracking system based on medication identification system 2. The medication delivery process steps can include data collection and tracking utilizing IV fluid containers and/or fluid transfer devices with ID Codes 40, 50 or 60. The 5-step process illustrated shows medication compounding, preparation/transfer and labeling in a pharmacy (Step 1), removal of medication 10 from an Automated Dispensing Station (ADS) 30 (Step 2), transfer of a partial volume of medication 10 from a primary source container 400 to a secondary container 600 (Step 3), administration of medication 10 in the secondary container 600 to a patient (Step 4), and wasting of residual medication 12 into a waste collection system 7 (Step 5). Initially, primary medication containers 4 can be filled by drug manufacturers and provided to pharmacies for use. These primary containers 4 can be used by pharmacies for custom compounding (by pharmaceutical compounding services or in-hospital pharmacies) in step 1 or directly stocked in medication dispensing stations and/or carts in step 2. In clinical practice, an IV fluid delivery process may involve more or fewer steps, or the order of steps many change. For example, compounding and labeling may occur following dispensing of a medication 10 from an ADS 30, and wasting of residual medication 12 may occur prior to administering a dose of medication 10 to a patient. A medication delivery process data collection and tracking system utilizing IV fluid containers and/or fluid transfer devices with ID Codes 40, 50 or 60 can work the same regardless of the number or order of process steps.

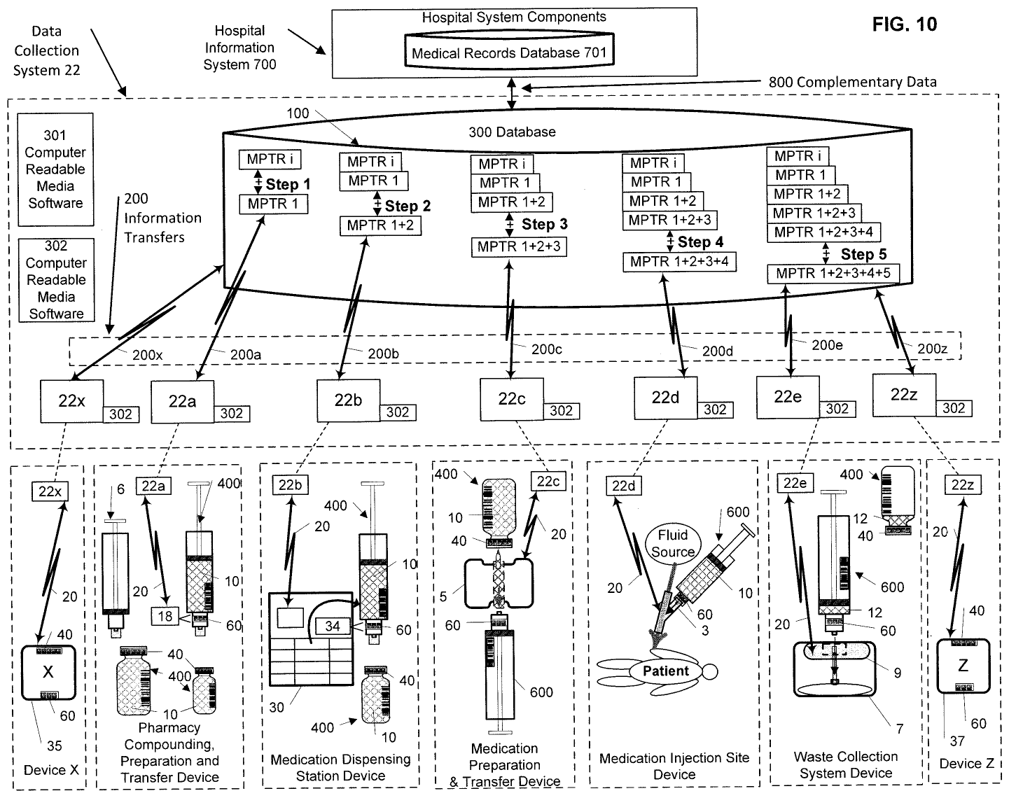

At the beginning of the first step of a medication delivery process, a Med Process Transaction Record (MPTR) data set 100 is created on data collection system 22. In FIG. 8, the data collection system 22 shown in each of the five process step drawings represents a single common system, which collects and processes information related to the medication delivery process. Data collection system 22 may include a database (shown and discussed later in FIG. 10). Data from devices and systems that interact with IV fluid containers and/or fluid transfer devices with ID Codes 40, 50 or 60 during Step 1 can be populated in the newly created MPTR 100. Each subsequent step following the first can add additional data to or can modify existing data within the MPTR 100. This data collection process continues until the medication delivery process is finished.

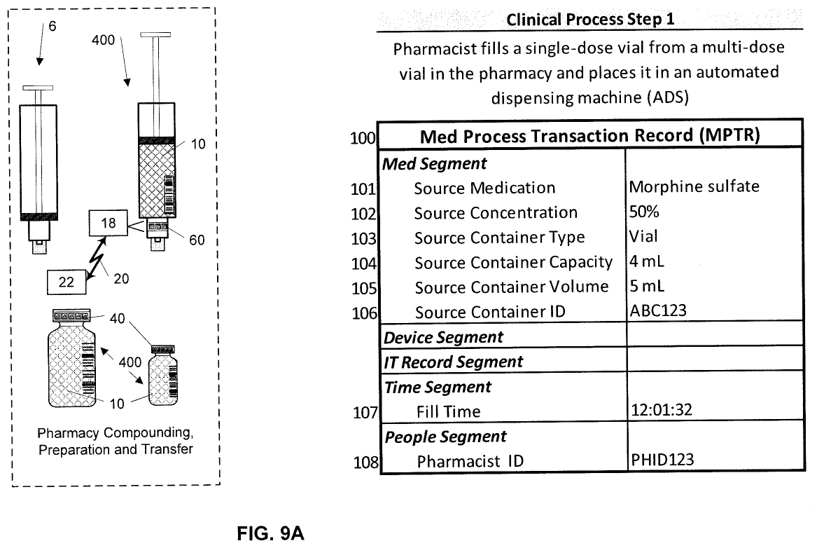

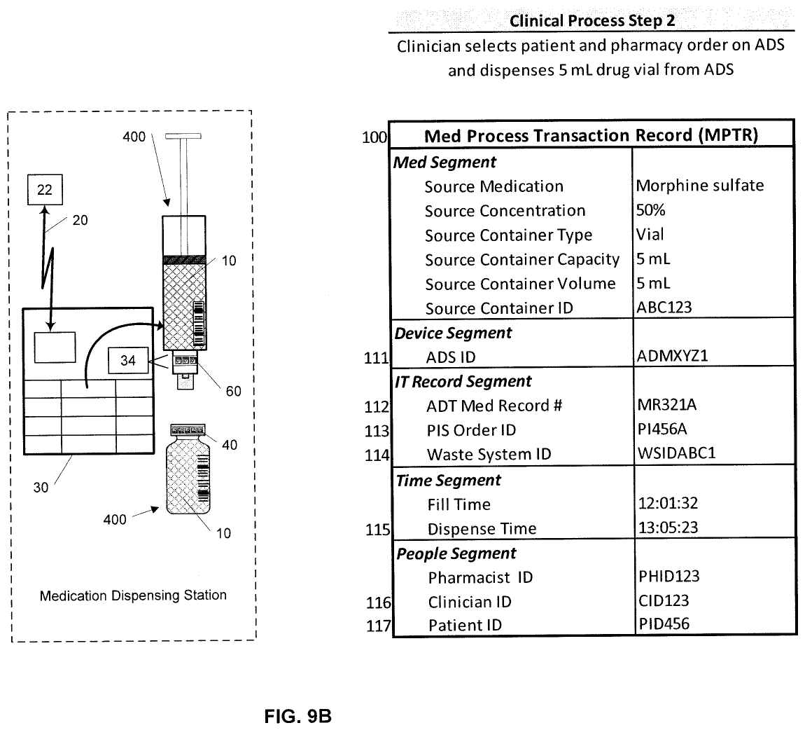

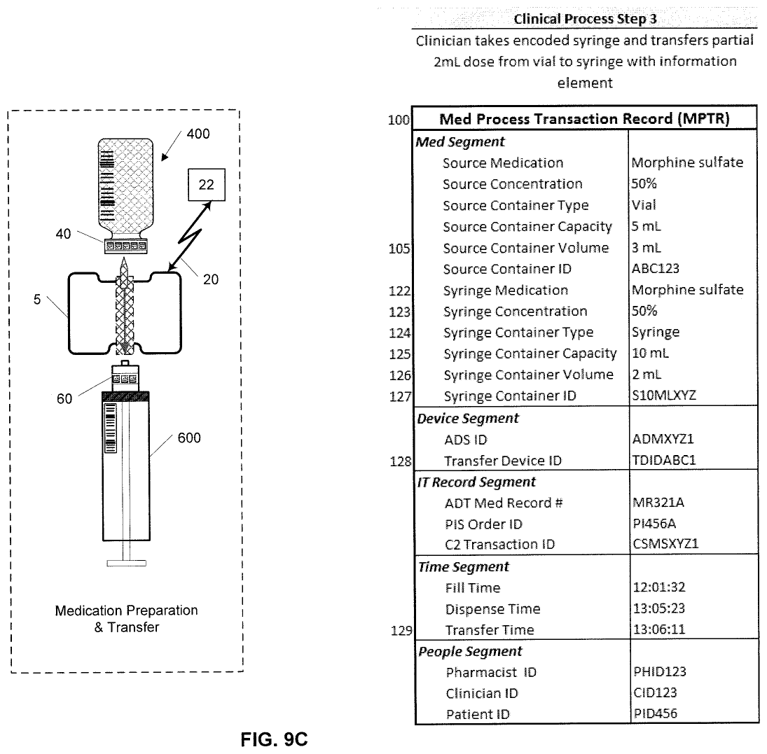

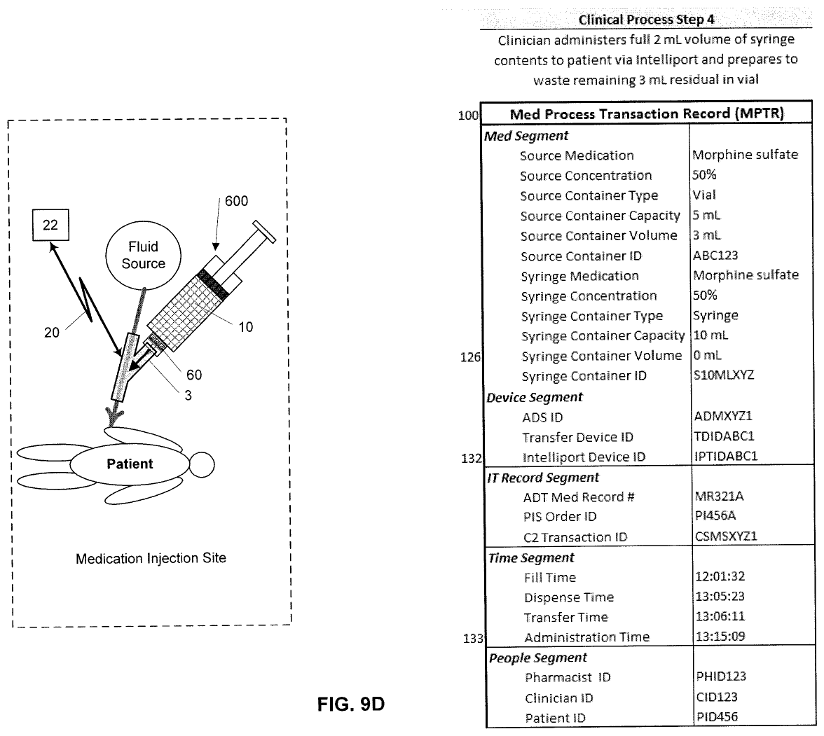

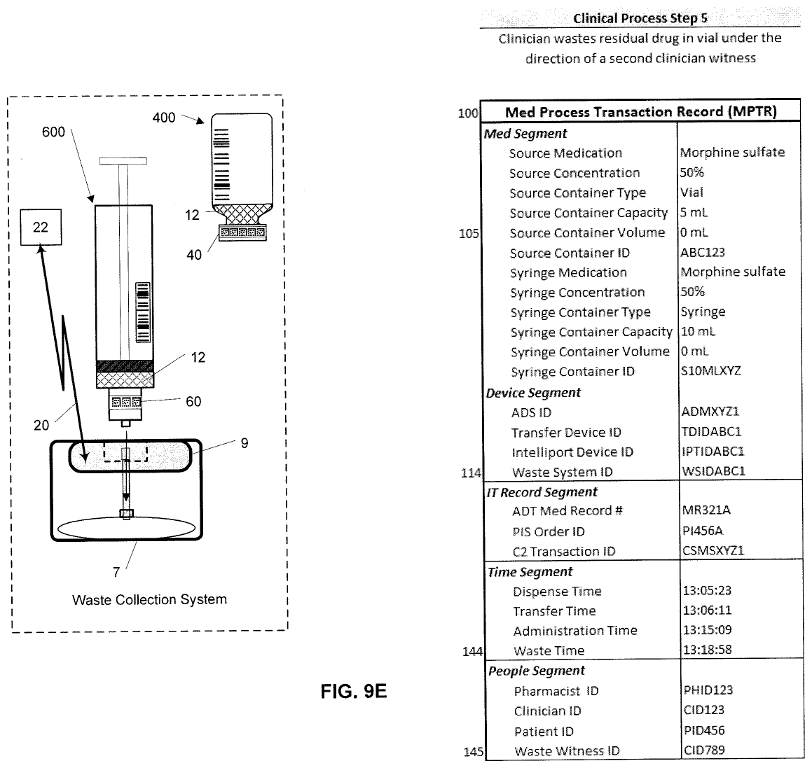

FIGS. 9 A-E illustrate using devices and systems to create a medication delivery process data set from a representative 5-step medication delivery process. FIG. 9A represents a medication preparation step (Step 1) where IV medication 10 is compounded in primary containers 400 which can include identification elements 40 (for vials) and 60 (for syringes) providing unique container identification information. During workflow process Step 1, MPTR 100 is created on data collection system 22 following the initial detection 18 of identifier 60 on container 400. Identifier detection can automatically initiate data transmission 20 to data collection system 22 and initialize MPTR 100. MPTR 100 is populated with information about the medication, time and people involved in workflow process Step 1, which in this example includes: source medication 101, source concentration 102, source container type 103, source container capacity 104, source container volume 105, source container ID 106, fill time 107, and pharmacist ID 108.

Continuing from the above example, in FIG. 9B, a clinician with ID “CID123” logs into a automated dispensing system 30 with ID “ADMXYZ1”, to dispense a medication for a patient with ID “PID456”, to fulfill a medication delivery order with ID number “PI456A” (workflow process Step 2). During dispensing, information from the patient's medical record which has number “MR321A” is accessed, and because the medication dispensed 10 is a controlled substance (morphine sulphate), a separate transaction record “CSMSXYZ1” is created on a waste collection system with ID “WSIDABC1”. While performing this step, the clinician can scan 34 identification element 60 (or 40 if a vial) to associate information related to the dispensing operation with the MPTR 100 created at the time medication container 400 was compounded in the pharmacy. When scan 34 occurs, information can be transferred 20 to data collection system 22 to add new data elements ADS ID 111, ADT med record #112, PIS order ID 113, waste system transaction ID 114, dispense time 115, clinician ID 116, and patient ID 117 to MPTR 100.

FIG. 9C illustrates workflow process Step 3 which represents a clinician transferring all or a partial volume of medication from a primary source container vial 400 with identification element 40 into a secondary syringe container 600 with identification element 60 using a dose preparation device 5. During medication transfer, information can be transferred 20 to data collection system 22 where it is used to update MPTR 100. In Step 3 new data elements including syringe medication 122, syringe concentration 123, syringe container type 124, syringe container capacity 125, syringe container volume 126, syringe container ID 127 (identification element 60), transfer device ID 128, and transfer time 129 can be added to MPTR 100 while the existing data element source container volume 105 can be modified to reflect the 2 mL of medication transferred from vial 400 to syringe 600.

FIG. 9D illustrates workflow process Step 4 which represents a clinician administering medication from a secondary syringe container 600 with information element 60 to a patient using medication injection site 3. During medication administration to the patient, information can be transferred 20 to data collection system 22 where it is used to update MPTR 100. In Step 4 new data elements Intelliport device ID 132 (medication injection site 3) and administration time 133 can be added to MPTR 100 while the existing data element syringe container volume 126 can be modified to reflect the 2 mL of medication administered from syringe 600 to the patient.

FIG. 9E illustrates workflow process Step 5 which represents a clinician wasting (disposing of) residual medication 12 from a secondary source container 600 with information element 60 to waste collection system 7 in the presence of a second clinician witness with clinician ID “CID789”. During the wasting of residual medication, intelligent injection site 9 can generate information that can be transferred 20 to data collection system 22 where it is used to update MPTR 100. In Step 5 new data elements including waste time 144 and waste witness ID 145 can be added to MPTR 100 while the existing data element source container volume 105 can be modified to reflect the 3 mL of residual medication 12 transferred from the source container 600 to the waste collection system 7. Alternatively, a primary container 400 with identification element 40 can be used to waste the medication using waste collection system 7.

FIG. 10 is a diagram further illustrating a record keeping system. Data collection system 22 can include remote device data sources (22x, 22a-e, 22z) that can collect data and provide user information local to a workflow process activity. Data collection 22 can include a centralized data base 300 forming a common record storage location. Data collection system 22 can include remote source devices (device 35, device 18, device 30, device 5, device 3, device 7, and device 37) that can transmit data 20 to the local data collection systems (22x, 22a, 22b, 22c, 22d, 22e, 22z respectively). In turn information can be transferred 200 (information transmissions 200x, 200a, 200b, 200c, 200d, 200e, 200z respectively) to a centralized database 300. Information can be requested (or polled) 20/200 by remote devices or systems from database 300, or hospital system database 700, for information associated with a medication container as a part of the container identification process. Additional data can be generated, modified, and/or appended to data set MPTR 100 as a result of data received from the request. The centralized database 300 can be provided with computer readable media software 301 to manage the MPTR database records. Computer readable media software 302 can also be provided to manage bi-directional information transfers/requests 200, remote source data collection (22x, 22a-e, 22z), remote device localized user interface data (audio-visual display of information in the form of: specific container identifier information, verification information, display of local data, display of MPTR records associated with container identifier 40, 50, 60, user identification, instruction messages, next step clinician guidance, precautionary messages, patient specific messages, medication specific messages, time specific messages) and many other types of clinical procedure and/or database record management information.

MPTR 100 data can be automatically initiated and/or transmitted 20 following detection of identifiers 40, 50, 60 by a remote data source device thus providing prompt and accurate record keeping and/or user information about the medication container without interruption of the normal medication workflow process. Each and every medication workflow process step can be tracked by generating, modifying, and/or appending MPTR 100. Workflow process steps can be added, subtracted, sequenced before or after others and time coordinated. Clinicians, healthcare providers and/or healthcare administrators can be informed of process steps to be completed, those already completed, and when they are to be completed or when they were completed. Clinicians and/or healthcare providers can also be prompted about what and when the next process step is to be completed, notified that medication administration documentation was provided confirming who/what/when/how much/and to whom medication (or any IV fluid) was administered. MPTR 100 can automatically provide patient and/or caregiver safety information relating to specific medication types and amounts to be or amounts that have been administered. Prompts and information provided to caregivers can be in the form of data displayed on, or visual and/or audio feedback provided by, a remote source device during or after detection of identifiers 40, 50, 60 by the corresponding remote source device.

Data collection system 22 can be linked to hospital system information system 700 to provide complementary data 800. Complementary data 800 can be fluid information, patient-specific information, medical order information, clinical guidance information, environmental information, historical patient information and other information stored in hospital information systems. System 700 can include many components and a medical records database 701. Computer readable media software 301 can provide an interface between data collection system 22 and hospital system 700 and can link database 300 with database 701. This linkage can form part of data collection system 22.

In addition to automated dispensing station 30, medication transfer device 5, injection site 3, waste collection system 7, and other remote source devices and systems (device 35 and/or device 37, for example) can add, modify and/or operate on data set MPTR 100. Device 35 can include the application of identification element 40, 50, 60 to primary container 4, secondary container 6 or fluid transfer device 16 at any step in the IV fluid delivery process including, but not limited to: the original pharmaceutical manufacturer's container characterization before, during or after a container filling operation; a medication container re-packager's (e.g. an external pharmacy compounding operation) container characterization before, during or after a re-filling operation; a hospital pharmacy's medication container characterization before, during or after a container filling operation; a medication or supply dispensing station withdrawal operation, during an IV fluid preparation and/or transfer to a secondary container, during an IV fluid administration to a patient, or during an IV fluid waste disposal done before or after an IV fluid administration to a patient. Data regarding the application and verification of identification element 40, 50 or 60 can be transmitted 20 to data collection system 22 as part container inspection, distribution, and/or inventory control. This data can be stored in database 701 and accessed by MTPR 100. Refer to “Medication Container Encoding, Verification and Identification”: U.S. patent application Ser. No. 13/149,782 filed on May 31, 2011.

Devices and systems can have one or more data processors and memory that can add, modify and/or operate on stored data set MPTR 100. Devices and systems can have one or more data processors that transmit data to and/or receive requested data from MPTR 100 data set. Computer readable media software 301 and 302 can include instructions for one or more of: receiving data characterizing a medication container; generating, modifying and/or appending MPTR 100 records stored in database 300 and/or 701; receiving requests for information relating to data associated with a characterized medication container and/or medication container identifier; transmitting MPTR 100 data set stored in database 300 or 701 to a remote source. Other devices and systems that can interact with MPTR 100 can include pharmacy information systems; inventory control systems, medication administration record systems; blood bank information systems; admissions, discharge and transfer systems; electronic medical record systems, infusion pumps, patient monitoring devices, anesthesia information systems, barcode verification systems, and automated supply cabinets, to list a few examples. Data set MPTR 100 can also reside within one of the aforementioned devices or systems, or be distributed across multiple devices or systems.

The data set MPTR 100 on data collection system 22 can be used for medical record documentation, process tracking, inventory control, drug diversion prevention, quality control measurement, statistical analysis, billing, compliance verification, or any other clinical or operational application of the data elements collected. Identification elements 40, 50 and/or 60 and data set MTPR 100 can provide information transfers 20 and/or 200 to accurately monitor, control and document medication preparation, administration and disposal.

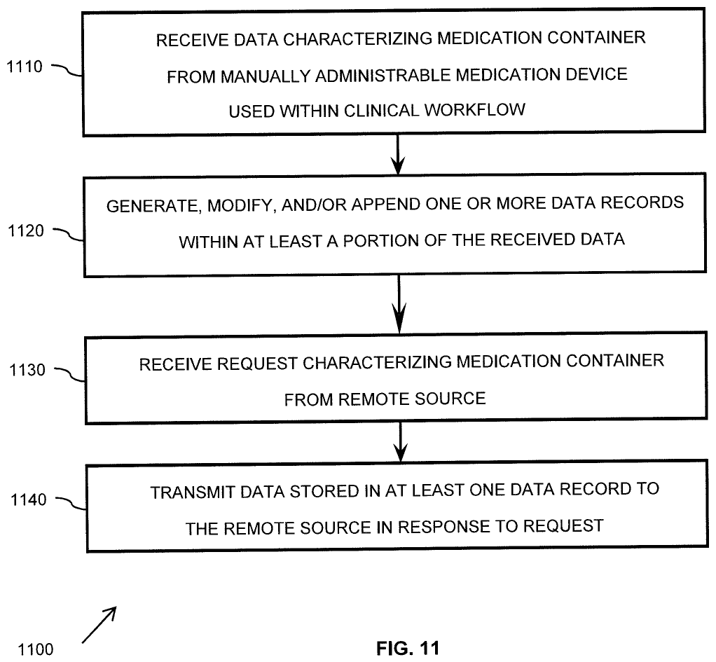

FIG. 11 is a process flow diagram 1100 in which, at 1110, data characterizing a medication container is received from a manually administrable medication device. The manually administrable medication device is one of a plurality of medical devices used within a clinical workflow and the medication container comprising an identifier. Thereafter, at 1120, at least one data record is generated, modified, and/or appended with at least a portion of the received data using the identifier. Later, at 1130, a request is received that includes the medication identifier from a remote source. In response to the request, at 1140, data stored within the at least one data record associated with the medication container is transmitted to the remote source.

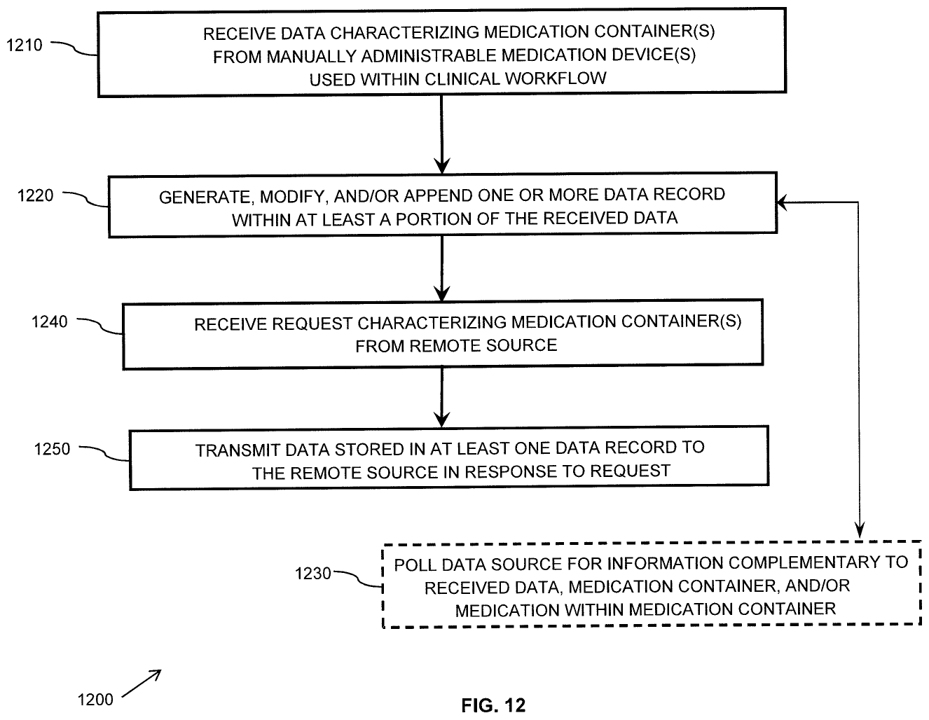

FIG. 12 is a process flow diagram 1200 in which, at 1210, data characterizing one or more medication containers is received from at least one manually administrable medication device. Each manually administrable medication device is one of a plurality of medical devices used within a clinical workflow and each medication container comprising an identifier. Thereafter, at 1220, at least one data record is generated, modified, and/or appended with at least a portion of the received data. In some optional variations, at 1230, at least one other data source is polled to obtain complementary data to the received data. This complementary data can be any data such as data corresponding to the medication container(s), data corresponding to medication contained within the medication container(s), data corresponding to a patient, data corresponding to instructions and/or guidance relating to caring for a patient, data corresponding to a caregiver, etc. Later, at 1240, a request is received from a remote source that characterizes a medication container or medication containers. In response to the request, at 1250, data stored within the at least one data record associated with the medication container(s) is transmitted to the remote source.

It will be appreciated that the remote sources requesting data about the medication container can be any of a wide variety of systems. Such remote sources can consume the transmitted data in a wide variety of fashions. For example, some or all of the transmitted data can be used to display information to a caregiver operating the remote source and/or provide audio or other visual feedback. Similarly, in cases such as with an intelligent fluid flow stop, the transmitted data can be used by a rules engine to determine whether or not to modify any operational parameters of any medical devices providing care to or monitoring the wellbeing of the patient or to take some other action (alert, etc.). In some cases, the remote source simply stores the transmitted data for subsequent consumption (either directly or by yet another device or system). Sample remote sources include, but are not limited to: pharmacy information systems, medication administration record systems, blood bank information systems, patient admissions record systems, electronic medical record systems, medical record documentation systems, anesthesia information management systems, operating room information systems, patient scheduling systems, barcode medication administration systems, barcode verification systems, clinical information systems, infusion pumps, patient-controlled analgesia systems, patient monitoring devices, automated medication dispensing systems, medication dispensing carts, automated supply cabinets, medication container filling units, medication compounding units, fluid composition sensors, medication preparation and transfer units, medication injection sites, intelligent fluid flow stops, medication waste and data collection systems, clinical procedure process tracking systems, inventory control systems, logistical tracking systems, drug diversion prevention systems, quality control measurement systems, statistical analysis systems, billing systems, and compliance verification systems.

Medical devices used within a clinical workflow for the care of a patient can include a wide variety of devices, including, but not limited to: a medication container filling unit, a medication compounding unit, a fluid composition sensor, an automated medication dispensing system, a medication dispensing cart, an infusion pump, a patient-controlled analgesia system, a medication preparation and transfer unit, a barcode medication administration system, a medication injection site, an intelligent fluid flow stop, and a medication waste collection system.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard or touch screen and/or a pointing device (e.g., a mouse, a touch screen, or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

What is claimed is:

1. A method comprising:
coupling a fluid outlet of a medication container to a medication port of a manually administrable medication device, wherein the manually administrable medication device includes a fluid flow stop disposed between the medication port and a fluid outlet configured to deliver fluid from the medication container to a fluid line leading to a patient;
automatically reading, with an identification sensor, information for the medication container including an identifier from an information transfer element on the medication container when the medication container is coupled to or in the process of being coupled to the manually administrable medication device;
transmitting, with a transmitter, the information for the medication container to a remote data collection system;
receiving, with at least one processor, data from or derived from a transmission from the manually administrable medication device that characterizes the medication container including the identifier for the medication container, the manually administrable medication device being one of a plurality of devices used within a clinical workflow;
generating, modifying, and/or appending, with at least one processor, at least one data record with at least a portion of the received data using the identifier;
receiving, with at least one processor, a request comprising the medication identifier from a remote source;
transmitting, with at least one processor, data stored within the at least one data record associated with the medication container to the remote source; and
selectively causing, with the remote source, the fluid flow stop to transition between a first state that prevents fluid flow and a second state that permits fluid flow based on the data stored within the at least one data record associated with the medication container.

2. The method as in claim 1, wherein the manually administrable medication device comprises a device to characterize manual actions of one or more individuals involved in filling or preparing to fill medication into the medication container, dispensing or preparing to dispense medication from the medication container, and wasting or preparing to waste medication from the medication container.

3. The method as in claim 1, wherein the remote source that generated the request is the manually administrable medication device that generated the received data.

4. The method as in claim 1, wherein the remote source that generated the request is one or more devices in the workflow other than the manually administrable medication device that generated the received data.

5. The method as in claim 1 further comprising: displaying, by the remote source, at least a portion of the transmitted data.

6. The method as in claim 1 further comprising: providing audio and/or visual feedback, by the remote source, relating to at least a portion of the transmitted data.

7. The method as in claim 1, further comprising:
applying at least one decision rule, by the remote source, using at least a portion of the transmitted data as input, the decision rule being used to determine how to provide care for a patient, characterize care given to the patient, and/or how to operate one or more medical devices within the clinical workflow.

8. The method as in claim 1, wherein the remote source comprises one or more systems selected from a group consisting of: pharmacy information systems, medication administration record systems, blood bank information systems, patient admissions record systems, electronic medical record systems, medical record documentation systems, anesthesia information management systems, operating room information systems, patient scheduling systems, barcode medication administration systems, barcode verification systems, clinical information systems, infusion pumps, patient-controlled analgesia systems, patient monitoring devices, automated medication dispensing systems, medication dispensing carts, automated supply cabinets, medication container filling units, medication compounding units, fluid composition sensors, medication preparation and transfer units, medication injection sites, medication waste and data collection systems, clinical procedure process tracking systems, inventory control systems, logistical tracking systems, drug diversion prevention systems, quality control measurement systems, statistical analysis systems, billing systems, and compliance verification systems.

9. The method as in claim 1, wherein the plurality of devices comprise one or more devices selected from a group consisting of: a medication container filling unit, a medication compounding unit, a fluid composition sensor, an automated medication dispensing station, a medication dispensing cart, an infusion pump, a patient-controlled analgesia system, a medication preparation and transfer unit, a barcode medication administration system, a medication injection site, and a medication waste collection system.

10. The method as in claim 1, wherein the received data comprises data selected from a group consisting of: a type of medication contained within the medication container, the concentration of medication contained within the medication container, a type of medication container, a maximum volume capacity of the medication container, a volume of medication contained within the medication container, a volume of medication extracted from the medication container, a volume of medication and/or diluents added to the medication container, a volume of medication manually administered from the medication container, a patient identifier, a caregiver identifier, a pharmacist identifier, a care area identifier, a pharmacy identifier, a device and/or system identifier, a medical order identifier, a primary container identifier, a secondary container identifier, a controlled substance identifier, at least one time stamp identifying the timing of an event within the clinical workflow, at least one medical procedure associated with the workflow, a medication expiration date, a dosage form of the medication, dose instructions for the medication, specific-patient administration instructions for a medication, a medication formulation, medication manufacturer information, a re-packager of the medication, a distributor of the medication, a medication package form, a medication package size, a medication container serial number, a medication lot number, a blood type of a patient, an NDC code (National Drug Code), an RxNorm code, a segment of an NDC code identifying a corresponding medication product, a segment of an NDC code identifying a corresponding medication package, a unique identifier code, a serialized NDC (sNDC) code, a drug classification, a human readable alphanumeric string, and a machine readable code.

11. The method as in claim 1, wherein the remote source comprises a second manually administrable medication device and the request is transmitted by such manually administrable medication device automatically upon reading of a medication container information transfer element.

12. The method as in claim 1 further comprising:
automatically transmitting, by the remote source, the request upon reading of the medication container information.

13. The method as in claim 1, wherein the identifier is one or more of: a unique number, a unique alphanumeric string, a unique symbol, or a uniform resource locator (URL).

14. The method as in claim 1, wherein the medication container identifier is linked to a secondary unique identifier.

15. The method as in claim 1, wherein the medication container is selected from a group consisting of: syringes, intravenous (IV) bags, disposable medication cartridges, disposable medication pouches, single and multi-dose vials, ampoules, and IV tubing.

16. The method as in claim 1, further comprising:
polling, with at least one processor, at least one data source for complementary data associated with at least one of the medication container and medication contained within the medication container; and
generating, modifying, and/or appending, with at least one processor, the at least one data record with complementary data received from the polling.

17. The method as in claim 16, wherein the complementary data comprises data selected from a group consisting of: fluid information, patient-specific information, medical order information, clinical guideline information, environmental factors, and historical patient information, a type of medication contained within the medication container, the concentration of medication contained within the medication container, a type of medication container, a maximum volume capacity of the medication container, a volume of medication contained within the medication container, a volume of medication extracted from the medication container, a volume of medication and/or diluents added to the medication container, a volume of medication manually administered from the medication container, a patient identifier, a caregiver identifier, a pharmacist identifier, a care area identifier, a pharmacy identifier, a device and/or system identifier, a medical order identifier, a primary container identifier, a secondary container identifier, a controlled substance identifier, at least one time stamp identifying the timing of an event within the clinical workflow, at least one medical procedure associated with the workflow, a medication expiration date, a dosage form of the medication, dose instructions for the medication, specific-patient administration instructions for a medication, a medication formulation, medication manufacturer information, a re-packager of the medication, a distributor of the medication, a medication package form, a medication package size, a medication container serial number, a medication lot number, a blood type of a patient, an NDC code (National Drug Code), an RxNorm code, a segment of an NDC code identifying a corresponding medication product, a segment of an NDC code identifying a corresponding medication package, a unique identifier code, a serialized NDC (sNDC) code, a drug classification, a human readable alphanumeric string, and a machine readable code.
